(19) United States
(12) Patent Application Publication
(10) Pub. No.: US 2005/0276758 A1
(43) Pub. Date: Dec. 15, 2005

(54) Method for screening agents against human prostate disease

(76) Inventors: Deborah J. Marshall, Blue Bell, PA (US); Linda A. Snyder, Pottstown, PA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/150,066

(22) Filed: Jun. 9, 2005

Related U.S. Application Data
(60) Provisional application No. 60/579,871, filed on Jun. 15, 2004.

(51) Int. Cl. A61K 49/00; A61K 48/00

(52) U.S. Cl. 424/9.2; 514/44

(57) ABSTRACT

Isolated polynucleotides encoding Cynomolgus monkey prostate specific antigen and polypeptides obtainable from the polynucleotides and uses are disclosed.

### CROSS-REFERENCE TO RELATED APPLICATIONS

This application claims the benefit of U.S. Provisional Application No. 60/579,871 filed 15 Jun. 2004, the entire contents of which is incorporated herein by reference.

### FIELD OF THE INVENTION

The present invention relates to Cynomolgus monkey prostate specific antigen and its uses.

### BACKGROUND OF THE INVENTION

Cancer is a serious disease that afflicts one in four people. In the last fifty years, there have been significant improvements in the early detection of cancer, as well as the development of a number of therapies to treat cancer. Therapies include surgery to remove primary tumors, and sublethal radiation and chemotherapy to treat disseminated disease. While these treatments have resulted in apparent cures for many patients, the treatments can be quite debilitating and are still often ineffective at preventing death from this disease.

Prostate cancer is the second leading cause of cancer-related death in men. Approximately 180,000 men will be diagnosed with prostate cancer each year and 40,000 succumb to the disease each year. Prostate tumor cells have a low proliferation rate and do not respond to standard chemotherapies, which are most toxic to the most rapidly dividing cells in the body. Instead, prostate cancer can be treated surgically, with radiation therapy or hormonal therapy. Surgery and radiation therapy can lead to undesirable side effects, such as incontinence and impotence. The disease can often be successfully managed with hormonal therapy, which starves the tumor cells for required growth factors. However, eventually all tumors treated in this way become androgen-independent and there is no effective treatment beyond that point.

Treatment of cancer with active immunotherapy has shown promise in many preclinical models, and in a few clinical trials as an alternative or adjunct to surgery, radiation or chemotherapy. The goal of active immunotherapy is to create a therapeutic immune response against tumor-specific antigens, which then targets tumor cells for destruction. However, most tumor antigens are self-antigens, to which the patient is tolerant. Indeed, central and peripheral tolerance mechanisms are expected to hamper the generation of effective immunity against tumors that express self-antigens.

One way to solve the problem of how to break tolerance against a given self-antigen is to use a closely related gene or protein from a different species as an immunogen. This type of immunization is also known as xenogeneic immunization and its potency lies in either the random creation of heteroclitic epitopes in the xenogeneic sequences with enhanced binding capacity to MHC class I antigens and/or the presence of strong helper epitopes within the xenogeneic sequence. For example, injection of plasmid DNA encoding a xenogeneic differentiation antigen is a powerful means to induce antibody and T-cell responses to otherwise poorly immunogenic self-antigens. This xenogeneic approach has been shown to work for a variety of cancer models using mouse or rat sequences by inducing active immunity to several different types of genes, including angiogenesis genes (Liu et al., Blood 102:1815-23, 2003), membrane glycoproteins (Wolchok et al., Cancer Immun. 1, 9-18 (2001); Sioud and Sorensen D., Eur. J. Immunol. 33, 38-45 (2003)), and integrins (Lou et al., Immunol. Invest. 31, 51-69 (2002)).

Prostate tumors and some breast malignancies express prostate specific antigen (PSA), also known as kallikrein 3 (KLK3), on their surface. PSA is well known as a serum marker for prostate cancer; increasing serum levels of PSA typically correlate well with the severity of the disease. It is unclear if PSA has a role in the etiology of prostate cancer; various reports have indicated that PSA could either enhance or inhibit tumorigenicity. Several cytotoxic T-lymphocyte (CTL) epitopes for PSA have been described for the HLA A2 and A3 haplotypes; identification of these epitopes support the possibility of generating therapeutic in vivo CTL by vaccination.

In fact, use of DNA encoding human and mouse prostate-specific membrane antigen (PSMA) has been tested in phase I clinical trials in patients with recurrent prostate cancer. See Wolchok et al., Semin. Oncol. 30, 659-66 (2003). These authors have also shown in pre-clinical studies that use of xenogeneic DNA (e.g., injection of human PSMA DNA into mice) is an absolute requirement to overcome immunologic tolerance.

The study of PSA as a potential target antigen for immunotherapy, as well as other studies investigating its physiologic role, has been hampered by the lack of appropriate animal models. There is no currently available mouse system to model the immunological aspects of prostate cancer, as no prostate-specific kallikrein gene (of which the PSA gene is a family member in primates) has been reported for mice. Indeed, none of the mouse kallikrein genes identified to date have the same expression pattern as the human PSA gene (Clements, J A, Endocr. Rev. 10, 393-419 (1989). The one exception to this has been the engineering of a transgenic mouse to express the human PSA gene (Wei et al., Proc Natl Acad Sci USA 94, 6369-6374 (1997). Thus, there is still a need for more relevant animal models for the treatment of human disease.

### BRIEF DESCRIPTION OF THE DRAWING

FIG. 1 shows the nucleotide and deduced amino acid sequence of full-length Cynomolgus monkey PSA.

FIG. 2 shows an amino acid sequence comparison of Cynomolgus monkey, Human and Rhesus monkey PSA.

### SUMMARY OF THE INVENTION

One aspect of the invention is an isolated polynucleotide comprising a polynucleotide having the sequence shown in SEQ ID NO: 1 or a complementary sequence, fragment or variant thereof.

Another aspect of the invention is an isolated polynucleotide comprising a polynucleotide having the sequence shown in SEQ ID NO: 2 or a complementary sequence, fragment or variant thereof.

Another aspect of the invention is an isolated polynucleotide comprising a polynucleotide having the sequence shown in SEQ ID NO: 4 or a complementary sequence, fragment or variant thereof.

Another aspect of the invention is an isolated polynucleotide comprising a polynucleotide encoding the amino acid sequence shown in SEQ ID NO: 3 or a complementary sequence, fragment or variant thereof.

Another aspect of the invention is an isolated polynucleotide comprising a polynucleotide encoding the amino acid sequence shown in SEQ ID NO: 5 or a complementary sequence, fragment or variant thereof.

Another aspect of the invention is an isolated polypeptide comprising a polypeptide having the sequence shown in SEQ ID NO: 3.

Another aspect of the invention is an isolated polypeptide comprising a polypeptide having the sequence shown in SEQ ID NO: 5.

Another aspect of the invention is an isolated full-length human prostate specific antigen consisting of the amino acid sequence shown in SEQ ID NO: 3.

Another aspect of the invention is an isolated mature human PSA consisting of the amino acid sequence shown in SEQ ID NO: 5.

Another aspect of the invention is a method of evaluating the efficacy of an agent used to treat diseases of the human prostate comprising administering the agent to a Cynomolgus monkey.

### DETAILED DESCRIPTION OF THE INVENTION

All publications, including but not limited to patents and patent applications, cited in this specification are herein incorporated by reference as though fully set forth.

As used herein, the term “DNA vaccines” or “nucleic acid vaccines” denotes compositions useful for the direct in vivo introduction of DNA encoding an antigen into tissues of a subject for expression of the antigen by tissue cells. DNA vaccines are described in, e.g., International Patent Publications WO 95/20660 and WO 93/19183.

As used herein, the term “nucleic acid adjuvant” means a nucleotide sequence coding for a protein or protein fragment that enhances an immune response to an antigen.

The present invention provides isolated Cynomolgus monkey (Macaca fascicularis) PSA polypeptides and polynucleotides. The invention also provides a full-length Cynomolgus monkey PSA having the amino acid sequence set forth in SEQ ID NO: 3 and the polynucleotide encoding it including, but not limited to, the polynucleotide having the sequence set forth in SEQ ID NO: 1 or 2 or their complementary sequences. Full-length Cynomolgus PSA is predicted to have a 24 residue N-terminal leader sequence (SEQ ID NO: 6). The invention also provides a mature Cynomolgus monkey PSA lacking the leader sequence and having the amino acid sequence set forth in SEQ ID NO: 5 and the polynucleotide encoding it including but not limited to, the polynucleotide having the sequence set forth in SEQ ID NO: 4 or its complementary sequences. The invention further provides for equivalent fragments and variants of Cynomolgus monkey PSA, as well as encoding or complementary nucleic acids, vectors comprising a Cynomolgus monkey PSA or fragments or variants, host cells containing such vectors and methods of making and methods of use of such Cynomolgus PSA, vectors or host cells.

A “fragment” is a polypeptide having an amino acid sequence that is part of but not all of any amino acid sequence of any polypeptide of the invention where the fragment contains residues 70 and/or 79 of the full-length form (equivalent to residues 21 and/or 46 of the mature form). Fragments can include, e.g., truncation polypeptides having a portion of an amino acid sequence as shown in amino acid sequence shown in SEQ ID NO: 3 or 5, or of variants thereof, such as a continuous series of residues that includes a heterologous amino- and/or carboxy-terminal amino acid sequence. Degradation forms of the polypeptides of the invention produced by or in a host cell are also included. Other exemplary fragments are characterized by structural or functional attributes such as fragments that comprise alpha-helix or alpha-helix forming regions, beta-sheet or beta-sheet forming regions, turn or turn-forming regions, coil or coil-forming regions, hydrophilic regions, hydrophobic regions, alpha-amphipathic regions, beta-amphipathic regions, flexible regions, surface-forming regions, substrate binding regions, extracellular regions and high antigenic index regions.

Specific exemplary fragments include residues 141 to 163 of the mature form that encode peptides that can bind human Class I molecules. See Correale et al., J. Natl. Cancer Inst. 89, 293-300 (1997) and Xue et al., The Prostate 30, 73-78 (1997). Other exemplary fragments include residues 16-25, 42-50, 48-56 and 75-83. Further exemplary fragments include an isolated polypeptide comprising an amino acid sequence having at least 10, 15, 20, 30, 40, 50 or 100 contiguous amino acids from the amino acid sequence set forth in SEQ ID NO: 3 where the fragment contains residues 45 and/or 70 of the full-length form, or an isolated polypeptide comprising an amino acid sequence having at least 10, 15, 20, 30, 40, 50 or 100 contiguous amino acids from the amino acid sequence set forth in SEQ ID NO: 5 where the fragment contains residues 21 and/or 46 of the mature form.

A “variant” polypeptide is a Cynomolgus PSA polypeptide or fragment in which amino acid substitutions, insertions, deletions or combinations thereof have been made. Naturally occurring, modified or atypical amino acids can be used for substitutions and insertions. A variant polynucleotide is a polynucleotide encoding variant polypeptides. Variant polypeptides of the invention elicit antimmune response to PSA in a host.

The polynucleotides of the invention are useful for preparing a composition having an isolated polynucleotide encoding an antigenic determinant of Cynomolgus monkey PSA and a promoter controlling expression of the polynucleotide. The composition can further include an isolated polynucleotide encoding a nucleic acid adjuvant such as interleukin-18 (IL-18) and a promoter polynucleotide controlling expression of the IL-18. These compositions can be used to elicit an immune response to a cancer-associated tumor protein in a mammal and are useful as nucleic acid vaccines for the treatment and/or prophylaxis of certain cancers or other tumor-related pathologies.

Antigenic determinants useful in the invention are obtained or derived from Cynomolgus monkey PSA. The tumor antigens could also be mutated to enhance their immunogenicity. Examples of how the antigen genes could be modified to effect a more robust immune response to the antigen protein include changes that affect antigen gene expression levels, such as addition of intron sequences, alteration or removal of signal sequences required for secretion or optimization of codons for improved translation. In addition, the antigen gene could be modified to introduce changes to the translated product of the gene, such as addition of ubiquitination signals for degradation, addition of subcellular compartment targeting sequences, addition of molecular chaperone sequences, and optimization of CTL epitope sequences. The antigen genes could be fused together to increase immunogenicity. The CTL/helper epitopes could be linked together, or inserted as part of another molecule, such as an immunoglobulin molecule. Nucleotides encoding at least one antigenic determinant of the molecules disclosed above are useful in the invention. Further, sequences complementary to any of the polynucleotides disclosed above are also useful in the compositions of the invention.

Compositions of the invention including these antigenic determinants are useful for the treatment of any cancer where PSA is uniquely expressed, over-expressed or associated with the presence of tumors caused by the cancer. These cancers include, but are not limited to, prostate, including hormone-refractory prostate cancer (HRPC), and breast cancer.

The invention also provides a mature polypeptide coding sequence or a fragment thereof in reading frame with another coding sequence produced synthetically or derived from another species, such as a sequence encoding a leader or secretory sequence, a pre- or pro- or prepro-protein sequence. The polynucleotides of the invention may also contain at least one non-coding sequence, such as transcribed but not translated sequences, termination signals, ribosome binding sites, mRNA stabilizing sequences, introns and polyadenylation signals. The polynucleotide sequences may also contain additional sequences encoding additional amino acids. These additional polynucleotide sequences may, for example, encode a marker sequence such as a hexa-histidine peptide, as described in Gentz et al., Proc. Natl. Acad. Sci. (USA) 86, 821-824 (1989) or the HA peptide tag as described in Wilson et al., Cell 37, 767 (1984) which facilitate the purification of fused polypeptides.

The invention also relates to vectors that comprise a polynucleotide or polynucleotides of the invention, host cells that are genetically engineered with vectors of the invention and the production of polypeptides of the invention by recombinant techniques. Cell-free translation systems can also be employed to produce such proteins using RNAs derived from the DNA constructs of the invention.

For recombinant production of the polypeptides of the invention, host cells can be genetically engineered to incorporate expression systems or portions thereof and polynucleotides of the invention. Introduction of a polynucleotide into a host cell can be effected by methods well known to those skilled in the art from laboratory manuals such as Davis et al., Basic Methods in Molecular Biology, 2^{nd} ed., Appleton & Lange, Norwalk, Conn. (1994) and Sambrook et al., Molecular Cloning: A Laboratory Manual, 3^{rd} ed., Cold Spring Harbor Laboratory Press, Cold Spring Harbor, N.Y. (2001). These methods include calcium phosphate transfection, DEAE-Dextran mediated transfection, microinjection, cationic lipid-mediated transfection, electroporation, transduction, scrape loading, ballistic introduction and infection.

Representative examples of hosts include Archaea cells; bacterial cells such as streptococci, staphylococci, enterococci, E. coli, streptomyces, cyanobacteria, B. subtilis and S. aureus; fungal cells such as Kluveromyces, Saccharomyces, Basidomycete, Candida albicans or Aspergillus; insect cells such as Drosophila S2 and Spodoptera Sf9; animal cells such as CHO, COS, HeLa, C127, 3T3, BHK, 293, CV-1, Bowes melanoma and myeloma; and plant cells, such as gymnosperm or angiosperm cells.

A great variety of expression systems can be used to produce the polypeptides of the invention. Such systems include chromosomal-, episomal- and virus-derived vectors such as vectors derived from bacterial plasmids, bacteriophage, transposons, yeast episomes, insertion elements, yeast chromosomal elements, baculoviruses, papova viruses such as SV40, vaccinia viruses, adenoviruses, fowl pox viruses, pseudorabies viruses, picronaviruses and retroviruses and vectors derived from combinations thereof, such as cosmids and phagemids. The expression system constructs may contain control regions that regulate or cause expression. Generally, any system or vector suitable to maintain or propagate polynucleotides and/or to express polypeptides in a host may be used for expression. An appropriate DNA sequence may be inserted into the expression system by any of a variety of techniques well known to those skilled in the art, such as, e.g., those set forth in Sambrook et al., supra.

In eukaryotic expression systems, polypeptides of the invention can be secreted into the lumen of the endoplasmic reticulum or extracellular environment by inclusion of appropriate secretion signals such as a signal peptide or leader sequence. These signals may be heterologous or endogenous to Cynomolgus PSA such as the signal sequence having the amin acid sequence shown in SEQ ID NO: 6 (predicted).

The polypeptides of the present invention may also be produced by chemical synthesis such as solid phase peptide synthesis on an automated peptide synthesizer, using known amino acid sequences or amino acid sequences derived from the DNA sequence of the polynucleotides of the invention. Such techniques are well known to those skilled in the art.

Polypeptides of the invention can be recovered and purified from recombinant cell cultures by well-known methods including ammonium sulfate or ethanol precipitation, acid extraction, high-performance liquid chromatography, anion or cation exchange chromatography, phosphocellulose chromatography, hydrophobic interaction chromatography, affinity chromatography, hydroxyapatite chromatography and lectin chromatography. Well-known techniques for refolding protein may be employed to regenerate an active conformation when the protein is denatured during isolation and/or purification.

The polynucleotides and polypeptides of the invention comprising at least one epitope of Cynomolgus PSA can be used to produce polyclonal or monoclonal antibodies. Techniques for making murine, chimeric, humanized and fully human monoclonal antibodies using protein or nucleic acid immunization are known to those skilled in the art.

The polynucleotides and polypeptides of the invention are also useful for assaying a medium for the presence of a substance that modulates PSA protein function by affecting the binding of a Cynomolgus PSA protein to serum proteins. Examples of modulators include polypeptides or small organic molecules.

For use as DNA vaccines, the polypeptides of the invention could be contained within one or more cellular delivery vectors such as plasmids, mammalian viruses, bacteria or mammalian cells having appropriate regulatory and control elements as are well known to those skilled in the art. For example, expression of the tumor antigen and nucleic acid adjuvant polynucleotide sequences could be under the control of a suitable promoter such as the human cytomegalovirus immediate early (HCMV IE) promoter or dihydrofolatereductase promoter and a polyadenylation (polyA) signal such as the SV40 late, SV40 early polyA signal or a synthetic polyA sequence. An intron may be included for enhanced expression, such as the HCMV IE intron A or natural introns from the antigen or adjuvant genes.

An exemplary plasmid useful with the polypeptides of the invention contains an E. coli origin of replication, an aph(3′)-1a kanamycin resistance gene, HCMV immediate early promoter with intron A, a synthetic polyA sequence and a bovine growth hormone terminator. Another exemplary plasmid contains an E. coli origin of replication, an ant(4′)-1a kanamycin resistance gene, Rous sarcoma virus long terminal repeat sequences, HCMV immediate early promoter and an SV40 late polyA sequence.

Examples of suitable viruses that can act as recombinant viral hosts for the polypeptides of the invention include vaccinia, canarypox, and adenovirus, as are known in the art. Various genetically engineered virus hosts (“recombinant viruses”) can also be used. Viral cellular delivery vectors containing the compositions of the invention can promote a suitable immune response that targets activation of B lymphocytes, helper T lymphocytes, and cytotoxic T lymphocytes.

A preferred recombinant virus for use with the compositions of the invention is vaccinia virus (International Patent Publication WO 87/06262; Cooney et al., Proc. Natl. Acad. Sci. USA 90, 1882-1886 (1993); Graham et al., J. Infect. Dis. 166, 244-252 (1992); McElrath et al., J. Infect. Dis. 169, 41-47 (1994)). In another embodiment, recombinant canarypox can be used (Pialoux et al., AIDS Res. Hum. Retroviruses 11, 373-381 (1995), erratum in AIDS Res. Hum. Retroviruses 11, 875 (1995); Andersson et al., J. Infect. Dis. 174, 977-985 (1996); Fries et al., Vaccine 14, 428-434 (1996); Gonczol et al., Vaccine 13, 1080-1085 (1995)). Another alternative is defective adenovirus or adeno-associated viruses or retroviruses (Gilardi-Hebenstreit et al., J. Gen. Virol. 71, 2425-2431 (1990); Prevec et al., J. Infect. Dis. 161, 27-30 (1990); Lubeck et al., Proc. Natl. Acad. Sci. USA 86, 6763-6767 (1989); Xiang et al., Virology 219, 220-227 (1996)). Other suitable viral vectors include attenuated or defective DNA virus, such as but not limited to herpes simplex virus (HSV) (see, e.g., Kaplitt et al., Molec. Cell. Neurosci. 2, 320-330 (1991)), papillomavirus, Epstein Barr virus (EBV), see, e.g., U.S. Pat. Nos. 5,990,091; 5,766,599; 5,756,103; 6,086,890; 6,274,147; 5,585,254; 6,140,114; 5,616,326; 6,099,847; 6,221,136; 6,086,891; 5,958,425; 5,744,143; 5,558,860; 5,266,489; 5,858,368; 5,795,872; 5,693,530; 6,020,172.

The polypeptides of the invention can be formulated in a pharmaceutically acceptable carrier or diluent. A variety of aqueous carriers may be employed, e.g., 0.4% saline, 0.3% glycine and the like. These solutions are sterile and generally free of particulate matter. These solutions may be sterilized by conventional, well known sterilization techniques (e.g., filtration). The compositions may contain pharmaceutically acceptable auxiliary substances as required to approximate physiological conditions, such as pH adjusting and buffering agents. The concentration of the polypeptides of the invention in such pharmaceutical formulation can vary widely, i.e., from less than about 0.5%, usually at or at least about 1% to as much as 15 or 20% by weight and will be selected primarily based on fluid volumes, viscosities and other factors, according to the particular mode of administration selected. Further, plasmids containing the polypeptides of the invention could also be formulated in microparticles or with lipid, buffer or other excipients.

The polypeptides of the invention can also be formulated with adjuvants that could aid delivery of DNA, maintain its integrity in vivo or enhance the immunogenicity of the vaccine. Chemical adjuvants can include compounds or mixtures that enhances the immune response to an antigen. A chemical adjuvant can serve as a tissue depot that slowly releases the antigen and also as a lymphoid system activator that non-specifically enhances the immune response (Hood et al., Immunology, 2nd ed., (1984), Benjamin/Cummings, Menlo Park, Calif., p. 384). Adjuvants include, but are not limited to, complete Freund's adjuvant, incomplete Freund's adjuvant, saponin, mineral gels such as aluminum hydroxide, surface active substances such as lysolecithin, pluronic polyols, polyanions, peptides, oil or hydrocarbon emulsions, keyhole limpet hemocyanins, dinitrophenol, and useful human adjuvants such as BCG (Bacillus Calmette-Guerin) and Corynebacterium parvum. Selection of an adjuvant depends on the subject to be vaccinated. Preferably, a pharmaceutically acceptable adjuvant is used. For example, a vaccine for a human should avoid oil or hydrocarbon emulsion adjuvants, including complete and incomplete Freund's adjuvant. One example of an adjuvant suitable for use with humans is alum (alumina gel). In a specific embodiment, compositions of the invention are administered intramuscularly in alum. Alternatively, the compositions of the invention can be administered subcutaneously, intradermally, intraperitoneally, intramuscularly or via other acceptable vaccine administration routes.

The polypeptides of the invention can be temporally administered in different orders or administered in different places in the body at the same time. The polypeptides of the invention could also be delivered by direct injection into muscle, skin, lymph node, or by application to mucosal surfaces. In a specific embodiment, polypeptides of the invention are provided intramuscularly. Other potential modes of delivery would include injection of DNA, followed by electroporation to enhance cellular uptake and expression of DNA or administration by a gene gun or a similar device. For screening anti-tumor activity of sera or cells from an individual immunized with a polypeptide of the invention, any suitable screening assay can be used, as is known in the art.

The polypeptides of the invention, when in a pharmaceutical preparation, can be present in unit dose forms. The appropriate therapeutically effective dose can be determined readily by those of skill in the art. A determined dose may, if necessary, be repeated at appropriate time intervals selected as appropriate by a physician during the treatment period.

The polypeptides of the invention can be lyophilized for storage and reconstituted in a suitable carrier prior to use. This technique has been shown to be effective with conventional protein preparations and art-known lyophilization and reconstitution techniques can be employed.

Another aspect of the invention is a method of evaluating the efficacy of an agent used to treat diseases of the human prostate comprising administering the agent to a Cynomolgus monkey. The high homology of PSA between humans and Cynomolgus monkey suggest that Cynomolgus is an appropriate model to test PSA-specific vaccines for prostate cancer, as well as drugs for treatment of prostate diseases, such as prostatitis. The agents useful in the method of the invention as vaccines include nucleic acids, polypeptides and cellular vaccines. The Cynomolgus prostate seems to be more like the human prostate than that of other primate species as assessed by anatomical and histological means (Habenicht et al., Prostate 11, 313-326 (1987)).

Success of therapeutic cancer vaccines will depend on the ability of the immune system to overcome tumor-induced immunosuppression or evasion of the immune response. The identification and sequencing of the cynomolgus PSA gene provides for the use of Cynomolgus monkey as a model to understand how a therapeutic vaccine may be able to break tolerance in a completely self-antigen setting. For example, animals can be immunized with a vaccine encoding the Cynomolgus PSA sequence, and evaluate the best way to break tolerance with vaccine modifications such as the inclusion of adjuvants, CpG oligodinucleotides or strong helper epitopes. This animal model will provide a system to gain insight into the mechanisms of nonresponsiveness to PSA and enable the development of methods for generating an autoimmune, cell-mediated response using PSA as the target antigen for the immunotherapy of human prostate cancer.

In another aspect of the invention, Cynomolgus is useful to test xenogeneic immunization strategies to boost the effective immune response against the PSA gene. Using the Cynomolgus PSA gene in active human immunotherapy could potentially provide a higher level of effectiveness to the current treatment modalities by potentially creating epitopes with enhanced binding capacity to MHC class I antigens or enhanced helper activities to stimulate antigen-specific T cell responses. To test this in a large animal model, human PSA vaccines can be tested in Cynomolgus monkeys to analyze the critical epitopes for this xenogeneic response.

The present invention will now be described with reference to the following specific, non-limiting examples.

### EXAMPLE 1

### Isolation, Cloning and Sequencing of Cynomolgus Monkey PSA Gene

RNA was purified from Cynomolgus prostate tissue (Cambrex Bio Science Walkersville Inc, Walkersville, Md.) using Trizol reagent (Invitrogen, Carlsbad, Calif.) according to the manufacturer's instructions. Reverse transcription reactions were carried out using Superscript II Reverse Transcriptase kit and were primed using an oligo dT primer. For polymerase chain reaction (PCR), a gene-specific forward primer that annealed to the translation initiation site was paired with a reverse primer that annealed to conserved regions within the 3′ untranslated regions of the Human and Rhesus PSA mRNAs (SEQ ID NOs: 11 and 12, Table 1, 5′ PSA and 3′ PSA). The first ATG is located at residues 19-21 of the 5′ PSA primer shown in Table 1. The 5′ PSA primer anneals to the translation initiation site with a BamHI site (underlined in Table 1) engineered on the 5′ end and the 3′ PSA primer anneals to a conserved region within the 3′ untranslated region with a BamHI site engineered on the 3′ end. PCR was performed using 100 ng of each primer with 100 ng cDNA template under the following conditions: 30 cycles of 94° C. 30 seconds, 60° C. 1 minute, 68° C. 1 minute, followed by one cycle of 68° C. for 5 minutes. Ten percent of the PCR reactions were analyzed on agarose gels, and revealed a band of approximately 1000 bp (data not shown). PCR products were purified using Qiaquick PCR purification (Qiagen, Valencia, Calif.). Approximately 60 ng of each product was directly sequenced using the Big Dye Terminator Cycle Sequencing Kit (PE Applied Biosystems, Foster City, Calif.), followed by analysis on a Prism ABI377 automated DNA sequencing apparatus. PCR fragments were sequenced using 20 ng of 5′ PSA seq and 3′ PSA seq primers corresponding to nucleotides 383-406 and 653-676, respectively (SEQ ID NOs: 13 and 14, Table 1). In addition, specific PCR products were also subcloned and sequenced. All PSA gene results were confirmed from prostate tissue from at least two Cynomolgus monkeys. The sequences of the fragments were compared using Vector NTi software (Invitrogen, Frederick, Md.) to the Genbank database and aligned with Human and Rhesus PSA sequences (Genbank accession #M21896 and #X73560, respectively). The cynomolgus PSA sequence was also BLAST searched against available embl and other sequence databases. BLASTP, TBLASTN and BLASTN searches were performed and no identical records were found.

To obtain the complete sequence of the 5′ and 3′ untranslated regions of the cynomolgus PSA gene, rapid amplification of cDNA ends (RACE) was employed. Total RNA was extracted from the prostates of at least two adult male Cynomolgus monkeys and the 5′ and 3′ untranslated regions of the mRNA transcript was amplified by ligase-mediated RACE reactions using the 5′ and 3′ RACE primers shown in Table 1 corresponding to nucleotides 49-72 and 782-805, respectively (SEQ ID NOs: 15 and 16).

TABLE 1

Cynomolgus PSA gene cloning and
sequencing oligonucleotides.

| | |
|---|---|
| Name | Sequence |

| | | |
|---|---|---|
| 5′ PSA | 5′-CTGGATCCCTGTGCCACCATGTGG-3′ | (SEQ ID NO: 11) |
| 3′ PSA | 5′-CCGGATCCTGCTGATTTCTTTTCC-3′ | (SEQ ID NO: 12) |
| 5′ PSA seq | 5′-CCAGCCACGACCTCATCCTGCTCC-3′ | (SEQ ID NO: 13) |
| 3′ PSA seq | 5′-CCCCAGAATCACCCGACAGGTGC-3′ | (SEQ ID NO: 14) |
| 5′ RACE | 5′-CGTCACGGACAGGGTGAGGAAGAC-3′ | (SEQ ID NO: 15) |
| 3′ RACE | 5′-GGAAGTGGAGCCAGGACACCATCA-3′ | (SEQ ID NO: 16) |

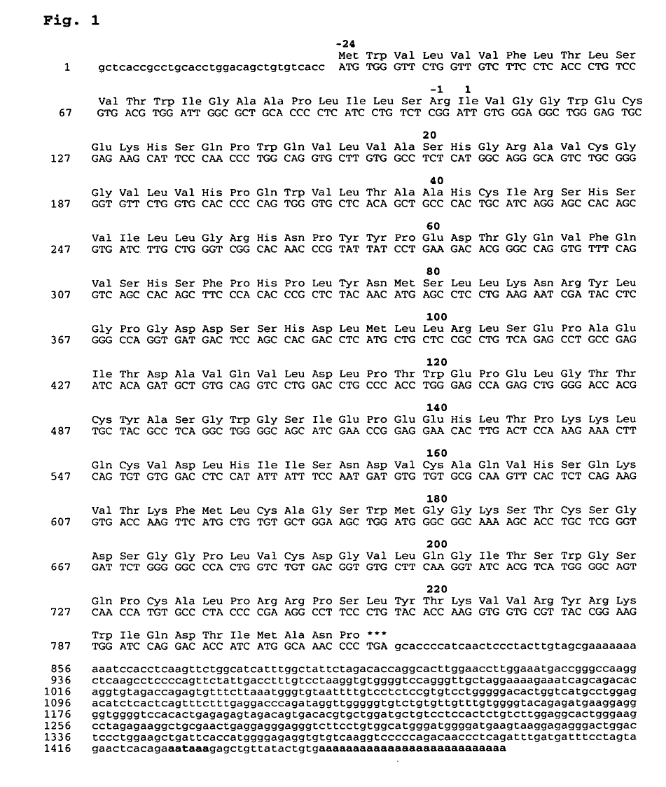

Four clones from three different monkeys were sequenced and aligned. The nucleotide and deduced amino acid sequence of full-length Cynomolgus monkey PSA cDNA is shown in FIG. 1 and SEQ ID NOs: 1 and 2. In FIG. 1, the translated amino acid sequences of the coding region are numbered and shown above the nucleotide sequence. The nucleotide residues are numbered on the left. The putative polyadenylation signal is in bold type.

Sequencing analysis of the cDNA fragments showed that the Cynomolgus PSA mRNA consists of a short 5′ noncoding region of about 33 bases (residues 1 to 33 of SEQ ID NO: 1), an open reading frame of 783 bases (residues 34 to 816 of SEQ ID NO: 1), corresponding to 261 amino acids (SEQ ID NO: 3) and a 3′ untranslated region of 660 bases (residues 817 to 1476 of SEQ ID NO: 1). The first 24 amino acids of Cynomolgus PSA are expected to include the signal sequence (SEQ ID NO: 6). The amino terminus of mature PSA is predicted to be the isoleucine shown as residue 1 in FIG. 1. The polynucleotide sequence and amino acid sequence of the mature 237 amino acid form of Cynomolgus PSA is shown in SEQ ID NOs: 4 and 5, respectively.

### EXAMPLE 2

### Sequence Comparisons of Primate PSA Genes

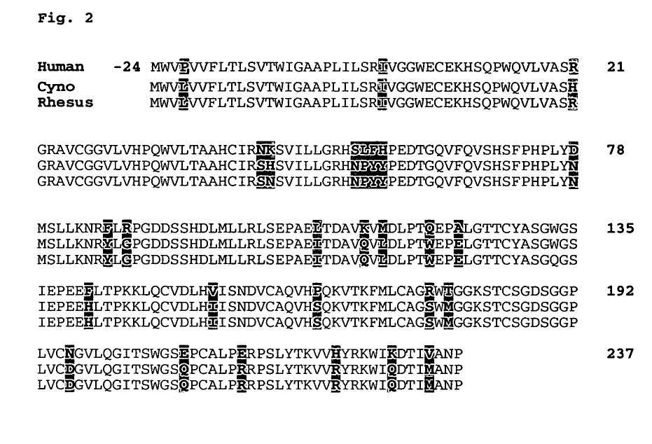

Protein sequence comparisons of the Cynomolgus PSA sequence to the Genbank database of Human and Rhesus PSA cDNAs are shown in FIG. 2. Residues that differ between the species are shown in bold and highlighted. Each species encodes a mature protein predicted to be 237 amino acids in length. The comparisons revealed that the Cynomolgus PSA amino acid sequence was 89.7% identical to Human PSA (27/261 residue differences) (FIG. 2). The Rhesus amino acid sequence was 90% identical to the human gene (26/261 residue differences). The Cynomolgus and Rhesus amino acid sequences differed at only two amino acids, namely residues 21 and 46 of the mature form, for 99.2% identity.

The nucleotide sequences of the three genes were also compared for the coding region. There is approximately 93.5% identity between Human and the Cynomolgus and Rhesus sequences (data not shown). Only three nucleotide residues differ in this region between the two primate cDNAs, giving 99.6% identity. Like the Human and Rhesus proteins, Cynomolgus PSA also has a serine at position 183, which has been shown to be responsible for the cleavage specificity of the serine proteases at hydrophobic amino acids (Lundwall, A. and Lilja, H. FEBS Letters 214, 317-322 (1987)).

The 5′ untranslated regions (UTR) were identical between Rhesus and Cynomolgus monkeys. The sequence of the Cynomolgus 5′ UTR was eight bases longer than the human sequence (data not shown).

There were several differences in the 3′ UTRs between the species. First, the Cynomolgus 3′ UTR is 75 bases shorter than the rhesus 3′UTR with a 72 base pair deletion located at bases 405/406 from the TGA stop codon of the Cynomolgus sequence. Besides these deletions, there are only three nucleotide differences in the 3′UTR between the species. As expected, there were more differences between the Human and Cynomolgus 3′UTR. First, there is a 54 base pair deletion in the human sequence corresponding to bp 34-87 of the Cynomolgus sequence. In addition, there are 4 base pair additions in the Cynomolgus sequence and one base pair deletion in addition to the 72 base pair deletion compared to the human sequence. There are also 31 base pair differences between the species (data not shown).

### EXAMPLE 3

### Analysis of PSA Expression in Cynomolgus Tissues

To investigate the RNA expression pattern of Cynomolgus PSA in Cynomolgus tissues, RT-PCR analysis was performed using RNA purified from different tissues. Total RNA from the kidney, lung, testis, thymus and prostate was isolated and cDNA was synthesized as described in Example 1. No DNA template was used as a negative control for non-specific amplification. Isolated total RNA was used as a template for first strand cDNA synthesis using Superscript II reverse transcriptase and an oligo dT primer. 100 ng of cDNA was subjected to PCR amplification as described above. The PSA gene was amplified using the 5′ PSA (SEQ ID NO: 11) and 3′ PSA (SEQ ID NO: 12) primers to yield a 1000 bp PSA-specific product. β-actin was amplified as a loading control using Cynomolgus-specific forward (SEQ ID NO: 17) and reverse (SEQ ID NO: 18) primers; a 216 bp product was amplified. The results indicated that PSA mRNA was only detected in the prostate tissue and not in the kidney, lung, testis or thymus (data not shown).

The present invention now being fully described, it will be apparent to one of ordinary skill in the art that many changes and modifications can be made thereto without departing from the spirit or scope of the appended claims.

1. A method of evaluating the efficacy of an agent used to treat diseases of the human prostate comprising administering the agent to a Cynomolgus monkey.

2. The method of claim 1 wherein the human prostate disease is prostate cancer.

3. The method of claim 1 wherein the human prostate disease is prostatitis.

4. The method of claim 2 wherein the agent comprises a vaccine.

5. The method of claim 4 wherein the vaccine is a DNA vaccine.

6. The method of claim 5 wherein the DNA vaccine comprises a polynucleotide encoding the amino acid sequence shown in SEQ ID NO: 3 or 5.


SEQUENCE LISTING (18 sequences)

SEQ ID NO: 1 - DNA, 1467 bp, Macaca fascicularis
gctcaccgcctgcacctggacagctgtgtcaccatgtgggttctggttgtcttcctcaccctgtccgtgacgtggattggcgctgcacccctcatcctgtctcggattgtgggaggctgggagtgcgagaagcattcccaaccctggcaggtgcttgtggcctctcatggcagggcagtctgcgggggtgttctggtgcacccccagtgggtgctcacagctgcccactgcatcaggagccacagcgtgatcttgctgggtcggcacaacccgtattatcctgaagacacgggccaggtgtttcaggtcagccacagcttcccacacccgctctacaacatgagcctcctgaagaatcgatacctcgggccaggtgatgactccagccacgacctcatgctgctccgcctgtcagagcctgccgagatcacagatgctgtgcaggtcctggacctgcccacctgggagccagagctggggaccacgtgctacgcctcaggctggggcagcatcgaaccggaggaacacttgactccaaagaaacttcagtgtgtggacctccatattatttccaatgatgtgtgtgcgcaagttcactctcagaaggtgaccaagttcatgctgtgtgctggaagctggatgggcggcaaaagcacctgctcgggtgattctgggggcccactggtctgtgacggtgtgcttcaaggtatcacgtcatggggcagtcaaccatgtgccctaccccgaaggccttccctgtacaccaaggtggtgcgttaccggaagtggatccaggacaccatcatggcaaacccctgagcaccccatcaactccctacttgtagcgaaaaaaaaaatccacctcaagttctggcatcatttggctattctagacaccaggcacttggaaccttggaaatgaccgggccaaggctcaagcctccccagttctattgacctttgtcctaaggtgtggggtccagggttgctaggaaaagaaatcagcagacacaggtgtagaccagagtgtttcttaaatgggtgtaattttgtcctctccgtgtcctgggggacactggtcatgcctggagacatctcactcagtttctttgaggacccagataggttgggggtgtctgtgttgtttgtggggtacagagatgaaggaggggtggggtccacactgagagagtagacagtgacacgtgctggatgctgtcctccactctgtcttggaggcactgggaagcctagagaaggctgcgaactgaggagggagggtcttcctgtggcatgggatggggatgaagtaaggagagggactggactccctggaagctgattcaccatggggagaggtgtgtcaaggtcccccagacaaccctcagatttgatgatttcctagtagaactcacagaaataaagagctgttatactgtgaaaaaaaaaaaaaaaaaaaaaaaaaaa

SEQ ID NO: 2 - DNA, 783 bp, Macaca fascicularis
atgtgggttctggttgtcttcctcaccctgtccgtgacgtggattggcgctgcacccctcatcctgtctcggattgtgggaggctgggagtgcgagaagcattcccaaccctggcaggtgcttgtggcctctcatggcagggcagtctgcgggggtgttctggtgcacccccagtgggtgctcacagctgcccactgcatcaggagccacagcgtgatcttgctgggtcggcacaacccgtattatcctgaagacacgggccaggtgtttcaggtcagccacagcttcccacacccgctctacaacatgagcctcctgaagaatcgatacctcgggccaggtgatgactccagccacgacctcatgctgctccgcctgtcagagcctgccgagatcacagatgctgtgcaggtcctggacctgcccacctgggagccagagctggggaccacgtgctacgcctcaggctggggcagcatcgaaccggaggaacacttgactccaaagaaacttcagtgtgtggacctccatattatttccaatgatgtgtgtgcgcaagttcactctcagaaggtgaccaagttcatgctgtgtgctggaagctggatgggcggcaaaagcacctgctcgggtgattctgggggcccactggtctgtgacggtgtgcttcaaggtatcacgtcatggggcagtcaaccatgtgccctaccccgaaggccttccctgtacaccaaggtggtgcgttaccggaagtggatccaggacaccatcatggcaaacccc

SEQ ID NO: 3 - protein, 261 aa, Macaca fascicularis
MWVLVVFLTLSVTWIGAAPLILSRIVGGWECEKHSQPWQVLVASHGRAVCGGVLVHPQWVLTAAHCIRSHSVILLGRHNPYYPEDTGQVFQVSHSFPHPLYNMSLLKNRYLGPGDDSSHDLMLLRLSEPAEITDAVQVLDLPTWEPELGTTCYASGWGSIEPEEHLTPKKLQCVDLHIISNDVCAQVHSQKVTKFMLCAGSWMGGKSTCSGDSGGPLVCDGVLQGITSWGSQPCALPRRPSLYTKVVRYRKWIQDTIMANP

SEQ ID NO: 4 - DNA, 711 bp, Macaca fascicularis
attgtgggaggctgggagtgcgagaagcattcccaaccctggcaggtgcttgtggcctctcatggcagggcagtctgcgggggtgttctggtgcacccccagtgggtgctcacagctgcccactgcatcaggagccacagcgtgatcttgctgggtcggcacaacccgtattatcctgaagacacgggccaggtgtttcaggtcagccacagcttcccacacccgctctacaacatgagcctcctgaagaatcgatacctcgggccaggtgatgactccagccacgacctcatgctgctccgcctgtcagagcctgccgagatcacagatgctgtgcaggtcctggacctgcccacctgggagccagagctggggaccacgtgctacgcctcaggctggggcagcatcgaaccggaggaacacttgactccaaagaaacttcagtgtgtggacctccatattatttccaatgatgtgtgtgcgcaagttcactctcagaaggtgaccaagttcatgctgtgtgctggaagctggatgggcggcaaaagcacctgctcgggtgattctgggggcccactggtctgtgacggtgtgcttcaaggtatcacgtcatggggcagtcaaccatgtgccctaccccgaaggccttccctgtacaccaaggtggtgcgttaccggaagtggatccaggacaccatcatggcaaacccc

SEQ ID NO: 5 - protein, 237 aa, Macaca fascicularis
IVGGWECEKHSQPWQVLVASHGRAVCGGVLVHPQWVLTAAHCIRSHSVILLGRHNPYYPEDTGQVFQVSHSFPHPLYNMSLLKNRYLGPGDDSSHDLMLLRLSEPAEITDAVQVLDLPTWEPELGTTCYASGWGSIEPEEHLTPKKLQCVDLHIISNDVCAQVHSQKVTKFMLCAGSWMGGKSTCSGDSGGPLVCDGVLQGITSWGSQPCALPRRPSLYTKVVRYRKWIQDTIMANP

SEQ ID NO: 6 - protein, 24 aa, Macaca fascicularis
MWVLVVFLTLSVTWIGAAPLILSR

SEQ ID NO: 7 - DNA, 783 bp, Homo sapiens
atgtgggtcccggttgtcttcctcaccctgtccgtgacgtggattggtgctgcacccctcatcctgtctcggattgtgggaggctgggagtgcgagaagcattcccaaccctggcaggtgcttgtggcctctcgtggcagggcagtctgcggcggtgttctggtgcacccccagtgggtcctcacagctgcccactgcatcaggaacaaaagcgtgatcttgctgggtcggcacagcctgtttcatcctgaagacacaggccaggtatttcaggtcagccacagcttcccacacccgctctacgatatgagcctcctgaagaatcgattcctcaggccaggtgatgactccagccacgacctcatgctgctccgcctgtcagagcctgccgagctcacggatgctgtgaaggtcatggacctgcccacccaggagccagcactggggaccacctgctacgcctcaggctggggcagcattgaaccagaggagttcttgaccccaaagaaacttcagtgtgtggacctccatgttatttccaatgacgtgtgtgcgcaagttcaccctcagaaggtgaccaagttcatgctgtgtgctggacgctggacagggggcaaaagcacctgctcgggtgattctgggggcccacttgtctgtaatggtgtgcttcaaggtatcacgtcatggggcagtgaaccatgtgccctgcccgaaaggccttccctgtacaccaaggtggtgcattaccggaagtggatcaaggacaccatcgtggccaacccc

SEQ ID NO: 8 - protein, 261 aa, Homo sapiens
MWVPVVFLTLSVTWIGAAPLILSRIVGGWECEKHSQPWQVLVASRGRAVCGGVLVHPQWVLTAAHCIRNKSVILLGRHSLFHPEDTGQVFQVSHSFPHPLYDMSLLKNRFLRPGDDSSHDLMLLRLSEPAELTDAVKVMDLPTQEPALGTTCYASGWGSIEPEEFLTPKKLQCVDLHVISNDVCAQVHPQKVTKFMLCAGRWTGGKSTCSGDSGGPLVCNGVLQGITSWGSEPCALPERPSLYTKVVHYRKWIKDTIVANP

SEQ ID NO: 9 - DNA, 783 bp, Macaca mulatta
atgtgggttctggttgtcttcctcaccctgtccgtgacgtggattggcgctgcacccctcatcctgtctcggattgtgggaggctgggagtgcgagaagcattcccaaccctggcaggtgcttgtggcctctcgtggcagggcagtctgcgggggtgttctggtgcacccccagtgggtcctcacagctgcccactgcatcaggagcaacagcgtgatcttgctgggtcggcacaacccgtattatcctgaagacacgggccaggtgtttcaggtcagccacagcttcccacacccgctctacaacatgagcctcctgaagaatcgatacctcgggccaggtgatgactccagccacgacctcatgctgctccgcctgtcagagcctgccgagatcacagatgctgtgcaggtcctggacctgcccacctgggagccagagctggggaccacgtgctacgcctcaggctggggcagcatcgaaccggaggaacacttgactccaaagaaacttcagtgtgtggacctccatattatttccaatgatgtgtgtgcgcaagttcactctcagaaggtgaccaagttcatgctgtgtgctggaagctggatgggcggcaaaagcacctgctcgggtgattctgggggcccactggtctgtgacggtgtgcttcaaggtatcacgtcatggggcagtcaaccatgtgccctaccccgaaggccttccctgtacaccaaggtggtgcgttaccggaagtggatccaggacaccatcatggcaaacccc

SEQ ID NO: 10 - protein, 261 aa, Macaca mulatta
MWVLVVFLTLSVTWIGAAPLILSRIVGGWECEKHSQPWQVLVASRGRAVCGGVLVHPQWVLTAAHCIRSNSVILLGRHNPYYPEDTGQVFQVSHSFPHPLYNMSLLKNRYLGPGDDSSHDLMLLRLSEPAEITDAVQVLDLPTWEPELGTTCYASGWGSIEPEEHLTPKKLQCVDLHIISNDVCAQVHSQKVTKFMLCAGSWMGGKSTCSGDSGGPLVCDGVLQGITSWGSQPCALPRRPSLYTKVVRYRKWIQDTIMANP

SEQ ID NO: 11 - DNA, 24 bp, Artificial
  Forward PCR primer, with an introduced BamHI site, which anneals to the translation initiation sites of the Homo sapiens and Macaca mulatta PSA cDNAs.
ctggatccctgtgccaccatgtgg

SEQ ID NO: 12 - DNA, 24 bp, Artificial
  Reverse PCR primer, with an introduced BamHI site, which anneals to a conserved region in the 3′ untranslated region of the Homo sapiens and Macaca mulatta PSA cDNAs.
ccggatcctgctgatttcttttcc

SEQ ID NO: 13 - DNA, 24 bp, Artificial
  Forward sequencing primer annealing to Macaca fascicularis PSA cDNA.
ccagccacgacctcatgctgctcc

SEQ ID NO: 14 - DNA, 23 bp, Artificial
  Reverse sequencing primer annealing to Macaca fascicularis PSA cDNA.
ccccagaatcacccgacaggtgc

SEQ ID NO: 15 - DNA, 24 bp, Artificial
  5′ RACE primer annealing to Macaca fascicularis PSA cDNA.
cgtcacggacagggtgaggaagac

SEQ ID NO: 16 - DNA, 24 bp, Artificial
  3′ RACE primer annealing to Macaca fascicularis PSA cDNA.
ggaagtggagccaggacaccatca

SEQ ID NO: 17 - DNA, 20 bp, Artificial
  Forward primer annealing to Macaca fascicularis PSA cDNA.
ccatgtacgtggccatccag

SEQ ID NO: 18 - DNA, 20 bp, Artificial
  Reverse primer annealing to Macaca fascicularis PSA cDNA.
tggtggtgaagctgtagccg